(19) United States
(12) Patent Application Publication
(10) Pub. No.: US 2021/0338897 A1
(43) Pub. Date: Nov. 4, 2021

(54) VASCULAR DEVICE AND METHOD FOR MANUFACTURING A VASCULAR DEVICE

(71) Applicant: Qvanteq AG, Zürich (CH)

(72) Inventors: Stefano Buzzi, Birmensdorf (CH); Armin Mäder, Richterswil (CH); Arik Zucker, Zürich (CH)

(21) Appl. No.: 17/270,242

(22) PCT Filed: Aug. 22, 2019
(86) PCT No.: PCT/EP2019/072427

§ 371 Date: Feb. 22, 2021

(30) Foreign Application Priority Data
Aug. 24, 2018 (CH) 01022/18

(51) Int. Cl. A61L 27/54; A61L 27/06; A61L 27/04; A61L 27/32; A61L 31/02; A61L 31/08; A61L 31/16; A61L 33/00; A61L 33/02

(52) U.S. Cl. CPC A61L 27/54; A61L 2430/36; A61L 27/045; A61L 27/32; A61L 31/022; A61L 31/086; A61L 31/16; A61L 33/0041; A61L 33/027; A61L 2300/112; A61L 2300/232; A61L 2300/42; A61L 2430/20; A61L 2430/22; A61L 27/06

(57) ABSTRACT

A vascular device for insertion in a body lumen, wherein the device includes a surface including at least a portion that is a functionalized surface provided with double or more charged ions such that the ions are exposed to a bodily fluid when the vascular device is inserted in the body lumen. The vascular device allows for reducing complications in its use and, particularly, for improving a desired healing in the body and preventing restenosis. At the same time, it allows for being manufactured at comparably low effort and for a convenient handling.

### CROSS-REFERENCE TO RELATED APPLICATIONS

The present application is the U.S. National Phase of PCT/EP2019/072427, filed on 22 Aug. 2019, which claims priority to Swiss Patent Application No. 01022/18, filed on 24 Aug. 2018, the entire contents of which are incorporated herein by reference.

### BACKGROUND

The present invention relates to a vascular device according to the preamble of independent claim 1 and more particularly to a method of manufacturing a vascular device. Such vascular devices having a surface to be exposed can be used as a prosthesis, e.g., in the form of a stent, to be positioned in a body lumen. In particular, vascular devices can be used in treatments of a broad variety of medical indications.

### RELATED ART

Vascular devices such as, e.g., stents inserted into body lumen such as, e.g., blood vessels for any medical purpose, entail certain risks for the patient. Among other things, adverse reactions such as e.g., inflammation or stenosis can occur in the body lumen. Stenosis can happen through thrombus formation on the surface of the vascular device or through neointimal hyperplasia.

Thereby, impurities on the surface of the vascular device, which can arise through usual handing and cleaning of the vascular device during the manufacturing process or during clinical application while transferring the vascular device from its packaging into the body, can influence the reaction of the body to the vascular device. Complications can be triggered through the adsorption of proteins on the surface of the vascular device as soon as the latter comes into contact with the body or respectively with a bodily fluid such as, e.g., blood inside the body lumen. The quantity, type and conformation of adhering proteins determine the further biological reactions between the body and the vascular device. The adsorption of certain components is thereby promoted or decreased and their effects activated or inhibited. This interaction between vascular device and body is decisive for the success or failure during the healing phase (sometimes also referred to as in-growth phase) of the vascular device in the body or for acceptance of the vascular device by the body.

The successful healing or setting of a vascular device, thus, depends on the characteristics and the condition of the surface of the implant. Known in the art are vascular devices with surface coatings, whereby the individual coatings are supposed to support and influence in one way or another the body reaction, i.e., acceptance, during the healing phase of the vascular device.

Also known are vascular devices with adapted surface structures. For example, EP 1 254 673 B1 describes a stent, the surface of which is provided in such a way that a recognition of the stent as foreign body is minimized. For this purpose, the surface structure of the stent is supposed to mimic the surface structure of the body's own cells. This is achieved by microstructures on the stent surface spaced from one another, which have an extension in a micrometer range. Stents of this kind are intended to exhibit an improved immunotolerance compared to stents with a smooth or generally rough surface. The healing phase of such stents can be further improved by a material being used with a positive surface charge, e.g., in the range of 0.03 to 0.05 N/m. The adhesion of fibrinogen on the stent surface can thereby be reduced. This is supposed to lead to a diminished inflammatory response and thereby decrease immune reactions.

Even though such vascular devices may provide some beneficial characteristics, they typically are costly in manufacture. Furthermore, vascular devices with such surfaces can make it difficult to dean the surface and keep it clean during handling in the manufacturing, storing and implantation process. Moreover, also with vascular devices of this kind, in some cases a reoccurring stenosis (restenosis) or other complications arise.

In this context, WO 2015/071322 A1 describes an implant with a surface, which is provided for contact with the body or a bodily fluid in the implanted state and which in a first state has a first surface charge. The characteristics of the implant surface in the first state, in particular the surface charge, can correspond to the features and the surface charge of a starting material, from which the implant is produced. The implant then undergoes a surface treatment, after which the surface of the implant assumes a second state with a second surface charge. The second surface charge is a lower positive surface charge or a higher negative surface charge compared to the first surface charge. Hence the surface in the second state, in which the implant is inserted into the body or a body lumen, has overall a more negative surface charge than in the first state.

The latter type of implants or vascular devices provides improved surface characteristics such that a better acceptance of the implant can be achieved. However, the second state of the surface typically is comparably instable such that measures have to be applied to prevent loss of the second state after manufacture of the implant. For example, it is known to store the implant inside a liquid filled container immediately after its manufacture in order to maintain the second state of the surface. Also, the surface treatment involved may cause a considerable effort in production.

Therefore, there is a need for a vascular device that reduces complications in use, in particular improving acceptance by the body in the healing phase and preventing thrombus formation and restenosis. At the same time, such vascular device is desired to be manufactured at comparably low effort and to allow for a convenient handling during manufacturing process as well as during clinical handling. It can further be an objective to improve an adsorption of proteins on the surface of the vascular device relating to tolerance of the implant towards the body and a successful implantation.

### SUMMARY

According to the invention, this need is settled by a vascular device as it is defined by the features of independent claim 1, by a method of manufacturing a vascular device as it is defined by the features of independent claim 20, and by a method of using a vascular device as defined by the features of independent claim 37, respectively. Preferred embodiments are subject of the dependent claims.

In one aspect, the invention is a vascular device for insertion in a body lumen. The vascular device comprises a surface, wherein at least a portion thereof is a functionalized surface provided with double or more charged ions such that the ions are exposed to a bodily fluid when the vascular device is inserted in the body lumen. The term “ion” as used herein relates to an electrically charged atom or molecule.

The term “body lumen” as used in connection with the invention can relate to an inside space of a tubular structure in a human or animal body or to a cavity inside the human or animal body. For example, the body lumen can be a vascular vessel, such as a vein or an artery, or a coronary or intracranial vessel, or a heart valve, or a tract of a gastrointestinal organ such as stomach or colon, or a region of urinary collecting ducts or of renal tubes, or of bile ducts, or of reproduction organs, or a tract of lung bronchi, or an eye's drainage system, or a cerebral spinal fluid (CSF) drainage, or an interior space of a joint, or a mouth or ear, or a combination thereof. It can also relate to an artificially made connection between two or more body lumina.

The term “vascular device” as used herein relates to any structure which may be temporarily or permanently positioned inside a body lumen of a human or animal being. For example, it can be an endodevice, being a device which is arranged or embodied to be introduced into the body lumen and to be advanced through it to a target location. At the target location the endodevice can perform functions such as imaging the tissue and/or executing an intervention to the tissue. The endodevice can be or comprise of a rigid or, particularly, a flexible endoscope, a catheter, a laparoscope, a colonoscope or a similar arrangement. The term “catheter” in this context can relate to essentially long tubes, e.g., made of a polymer material, that are inserted into the body lumen. These catheters can be used to steer or guide implants mounted on their delivery systems through body lumens towards an implantation site. Or, catheters can be implant delivery systems such as balloon catheters to deliver a balloon expandable stent or a tube catheter to deliver a self-expanding stent, or a wire onto which an implant is attached or just a balloon catheter without a stent. Or, the vascular device can also be any sort of tube such as a drainage system that is inserted into the body lumen. Or, it can be a cardiac pacemaker or the like.

More particularly, the vascular device can be an implant for insertion in the body lumen. When inserted, such implant typically contacts at least partially a wall of the body lumen such as, e.g., a tubular structure or blood vessel and/or contacts a bodily fluid, which follows the body lumen such as, e.g., blood. Thereby, implants typically comprise a plain, smooth, unroughened surface unlike what is common for implants for bones, for example. Such a plain surface may also lack—or may have been purposely deprived of—any substantial roughness, or at least any roughness at an interior surface of the implant, or waviness of its topology or any substantial texture. This may prove especially advantageous for supporting a thorough cleaning or purification of the implants and, conversely, for preventing contamination from the environment such as ambient atmosphere, from storage means or from manipulation. This is the case for bare metal stents, by way of example, wherein additionally no coating for elution with active substances may be envisaged. In this sense, the preferred, optional absence of specific treatments for integrating a complicated roughness or peak and valley topography in the implants may prove beneficial to making the implants less susceptible to contamination and to formation of thrombi.

The term “plain” as associated with possible embodiments of the implant or vascular device can indicate a substantially smooth surface whose roughness is comprised in a range of up to 10 micrometer or advantageously up to 5 micrometer. Such a plain surface also poses special requirements with respect to the material of a surface sealing as described below, which must be able to adhere to such a plain surface.

The implants being vascular devices in accordance with the invention advantageously are elastic and flexible, enabling an adjustment to the shape of the body lumen by deformation. Such a deformation can be induced, for instance, in the case of a stent used in a balloon angioplasty procedure. Or, it can happen by using memory shape materials and/or braided filaments, such as in the case of a self-expanding stent.

In a preferred embodiment, the vascular device is a vascular stent, e.g., formed by braided, knitted or woven structures, or by laser cutting, a flow diverter, e.g., for treating bifurcation aneurysm, an ocular stent, a coil or web-like structure for the treatment of vascular aneurysm, a heart valve, a cage of a heart valve, a part of a cardiac pacemaker such as an electrode, a flow disruptor such as a coil, a web or web-like coil, a neck bridging device, an intra-aneurysmal stent, an occluder, an adjustable remodelling mesh, an aneurism dip, a vena cava filter or other filter used during a clinical intervention, or a shunt. In the case of cardiac electrodes, the body lumen can be the pericardium. The vascular stents can be intracranial stents, coronary stents, endovascular/peripheral arterial stents, or endovascular/peripheral venous stents, or shunts such as cerebral spinal fluid (CSF) shunt systems.

The surface of the vascular device can particularly be an outer surface of it, an inner surface of it, or a combination of at least of portion of both surfaces. The outer surface is considered to mainly face towards the wall of a body lumen. The inner surface is considered to mainly not face the wall of the body lumen. The at least a portion of the surface can be the complete outer, the complete inner, or the complete total surface of the vascular device or a portion thereof, particularly, a substantial portion such as at least 50%, 70% or 90% of the complete surface. When being provided with the ions, the at least a portion of the surface is functionalized. Thus, the at least a portion of the surface together with the ions is referred to as the functionalized surface. Advantageously, the functionalized surface is hydrophilic. Preferably, the at least a portion of the surface is a contact surface, which is configured to contact the bodily fluid when the vascular device is inserted in the body lumen. Hence, this contact surface can be the outer surface, the inner surface, or a combination thereof.

By providing double or more charged ions to the at least a portion of the surface of the vascular device and thereby establishing the functionalized surface, the risk of thrombus formation that can lead to restenosis can be essentially reduced and acceptance of the vascular device by the body can be essentially increased. It has been shown, that by exposing the vascular device with the ions, instead of the bare metal surface, to the body lumen or, particularly, a bodily fluid circulating therein, thrombogenicity can be significantly reduced. Thus, provision of the double or more charged ions allows for functionalizing the at least a portion of the surface.

The vascular device having such functionalized surface can additionally improve adsorption of proteins on the surface. This may essentially increase tolerance or acceptance of the vascular device by the body it is inserted to such that the chance of a successful implantation or insertion is increased.

Furthermore, vascular devices having functionalized surfaces in accordance with the invention can comparably easily be manufactured. As described in more detail below, no complicated process has to be involved and no sophisticated substances are required. Uke this, the vascular device having the mentioned advantageous properties can efficiently be manufactured at comparably low costs.

Thus, the vascular device according to the invention allows for reducing complications in its use. In particular, it allows for improving a desired acceptance in the body and preventing restenosis or the formation of thrombi. At the same time, it allows for being manufactured at comparably low effort and for a convenient handling throughout manufacturing and deployment in the patient.

Generally, the double or more charged ions (i.e., ions that can assume double or more charge between pH 1 and 14 even though they might only have a single charge in a certain pH range, including zwitterions) can be any type of ions. It can include metallic cations, phosphates, carbonates, sulfates, borates, organic acids or any combination of the above. It can also comprise chemical combinations of the above or together with other ionic groups, such as nitrates, amines, leading to molecules with more than one charged group, for example, phosphorylcholine. Depending on the surface charge of the metal alloy, cations or anions at a particular pH result in favourable combination. In one preferred implementation, the ions are anions comprising phosphate, sulfate, borate or carbonate groups or organic acids, any combination thereof, or molecules with more than one charged group such as respective organic molecules. Such anions have been shown to be particularly advantageous in functionalizing the at least a portion of the surface according to the invention. Especially, Phosphate ions may be beneficial in many applications of the vascular device as Phosphate and Phosphate containing molecules are abundant in biological organisms. Also, Phosphate ions may result in beneficial reaction products when the vascular device is implanted. For example, when the vascular device is implanted or inserted, the Phosphate ions may react with bivalent ions such as Calcium ions contained in a natural or simulated bodily fluid. Thereby, the surface of the vascular device can be provided with particular antithrombotic properties. As specific example, the ions preferably are Phosphate ions, and the surface of the vascular device is made of Titanium or Nickel-Titanium alloys, referred to as Nitinol.

The at least a portion of the surface or the complete surface can be made of Titanium, a Titanium alloy such as Nitinol, Stainless Steel, a Chromium alloy such as Cobalt-Chromium or Platinum-Chromium, Platinum-Chromium, Titanium, a Titanium alloy, Zirconium oxide, a Zirconium containing alloy, or a polymeric plastic. More specifically, in preferred embodiments, the surface or the complete surface is made of Titanium, a Titanium alloy such as Nitinol, a Chromium alloy such as Cobalt-Chromium or Platinum-Chromium, Tantalum, Platinum, or Zirconium oxide. Such materials are advantageous in applications involving introduction into a body lumen. Particularly, such materials can be efficiently prepared to have desired properties and functionalized by the provision of the double or more charged ions. More specifically, it has been shown that for surfaces made of Nitinol Phosphate can be a very well suitable double or more charged ion to be provided on the surface.

Providing the double or more charged ions to the at least a portion of the surface, preferably results in the ions being bound to the at least a portion of the surface. Particularly, the ions can be chemically bound such that the functionalization of the surface can be efficiently maintained during handling and insertion or implantation of the vascular device. Like this, the vascular device can keep its characteristics to achieve a reduced tendency to restenosis or thrombotic reactions after implantation or insertion for a comparably long time, such as over its complete lifecycle. For matter of completeness it is to note that, when being bound to the at least a portion of the surface, the ions may not expose the complete original charge. For example, double ions provided to the at least a portion of the surface may be bound such that only a first charge is used for the binding and a second charge is exposed to the bodily fluid when the vascular device is inserted in the body lumen.

In a preferred embodiment, at least a portion of the functionalized surface is entirely or at least partially covered with a surface sealing which is soluble when inserting the vascular device in the body lumen. By providing the functionalized surface with the surface sealing, which covers this portion of the implant surface and is soluble when the vascular device is inserted in the body lumen, the functionalized surface can be protected and maintained until the vascular device is applied. Advantageously, the functionalized surface of the vascular device covered by the surface sealing is exposed to the body lumen during insertion such that the surface sealing can be dissolved. Thus, it can be guaranteed that the vascular device is placed in the body lumen with a plain, functionalized and hydrophilic surface which is optimally preserved by the surface sealing. Like this, the vascular device can best cooperate with a tissue wall and/or with bodily fluid of the body lumen, particularly in a way to be free of thrombosis and/or restenosis. The quality of the surface can be maintained while the implant is stored or supplied under dry conditions and while being handled during preparation of its insertion. Such preservation can be particularly important for hydrophilic stents or similar implants, of which the surfaces are highly purified and/or highly hydrophilic and quickly become re-contaminated.

In other words, the surface sealing can dissolve shortly before, during or after insertion in the body lumen, for instance upon contact with the bodily fluid passing though the body lumen. Thus, the function of shielding the defined surface characteristics remains effective at least from applying the surface sealing during production throughout packaging, sterilization, storage, transport and unpacking. As soon as the implant is prepared and ready to be inserted into the body lumen, the function of shielding the surface is not necessary anymore and the surface sealing can be dissolved at this time or at least shortly after insertion of the vascular device in the body lumen. The surface of the vascular device received in, or on, the given body lumen is consequently in its best condition to ensure success of treatment.

The term “when inserting the vascular device in the body lumen” in the context of the invention relates to the time frame where all necessary steps involved with introducing the vascular device into the body are performed. This may at least include preparatory steps to get the vascular device ready to be ushered into the body, steps of transferring and transporting the vascular device within the body, and further preparatory steps before final insertion to or implantation at the target location to fulfil the intended function. In particular, it may include the step of unpacking the vascular device, preparing the vascular device for providing it through an opening in the body, providing it through the opening in the body and forwarding it inside the body to a target location until shortly before placing it at the target location. Thus, the surface sealing can be dissolved as early as during unpacking and preparing the vascular device, e.g., by flushing it before providing it through the opening in the body. It can also be dissolved while being provided through an opening in the body and forwarded inside the body until the target location is reached. In case of a stent, it can be dissolved at latest shortly before expanding it at the target location.

The surface sealing can be designed to dissolve upon contact with bodily fluids flowing through the body lumen, without a need for preventive flushing or partial or total removal. For example, it can be dissolved in blood. Alternatively, it can also be devised to be flushable with a solvent (e.g., saline solution) just before insertion of the vascular device in the body lumen.

As mentioned, by the surface sealing, target properties which have been previously created or pre-assigned on the surface of the vascular device can be well preserved, at least up to when the device is brought into the body lumen. In particular, a comparably high hydrophilicity, which may also be implemented by the functionalization of the at least a portion of the surface and which reduces thrombogenicity due to lower adhesion of platelets on hydrophilic surfaces, can be maintained or preserved, as well as the above mentioned functionalization by the ions. For many applications, such pre-assigned target surface properties/functionalization advantageously comprise antithrombotic properties to prevent build-up of thrombi, adjusted surface charge properties in order to selectively regulate protein deposits and/or to prevent contaminant hydrocarbon deposits, as well as hydrophilicity to foster frictionless accurate implant insertion and therefore limiting potential tissue damage and early tissue healing. Also, functionalization with ions according to the invention and/or a medicament dispensing coating can be protected by the surface sealing.

The surface sealing preferably is configured to dissolve within 30 seconds (s) or within 20 s, or within 10 s. It is aimed that the surface sealing is dissolved as soon as it comes into contact with blood or washing buffer or other liquid solution that is used for device flushing for preparation. When being inserted into the body lumen or during flushing prior to insertion, it is advantageous to quickly dissolve the surface sealing such that the pure and functionalized surface can be exposed as directly upon insertion of the vascular device. Particularly, in case of an expandable or reshapeable implant such as a stent, the implant also has to remain flexible during the placement procedure. In such a case, the surface sealing that does not dissolve upon first contact with blood or washing buffer could have a negative impact on deliverability, in particular, for self-expanding structures where the undissolved sealing can lock the stent in a certain conformation. A fast dissolving surface sealing may also reduce the exposure of the patient to foreign substances.

As mentioned above, the surface sealing advantageously is configured to be dissolved when being inserted and positioned at a target location in the body lumen. In particular, the surface sealing can be configured to be dissolved when arriving at the target location or before being implanted at the target location. Thereby, it can be configured in accordance with a predefined insertion procedure such that it can be assured that the surface sealing is dissolved in time. The term “Implanting” as used in this connection can relate to securing the implant at or in the body lumen at the target location. For example, in case of a balloon- or self-expandable stent, implanting or implantation can be or involve the expansion of the stent at the target location. Such a surface sealing allows for preserving original mechanical properties of the implant, such as its expansion properties for example, even though its surface is protected by the surface sealing before and eventually while inserting it. This can be important, for example, when stents or other expandable implants are involved, wherein proper expansion of the implant potentially can be affected by the surface sealing. In case of a non-expanding stent, such as e.g., an ocular stent, implanting or implantation can be or involve the placement of the stent at the target location.

The sealing should not interact with the stent surface (bare, functionalized or else modified). If the sealing of a functionalized surface does not contain the ions used to functionalize the surface, for example, such sealing may deteriorate the properties of the surface during application and curing of the sealing, as well as during storage. For preventing such effects, the surface sealing preferably also comprises ions. Thereby, the ions (and pH) of the surface sealing preferably are of the same kind as the ions used to functionalize the at least a portion of the surface. Preferably, the surface sealing is provided with double or more charged ions. Such ions in the surface sealing may prevent or reduce transition of the ions between the at least a portion of the surface and the sealing. More specifically, when an ionic disbalance between the surface and the surface sealing is given, there may be a tendency to equilibrate the ion distribution between the surface and the surface sealing. This can result in a transfer of ions from the surface into the sealing, which may affect the functionalization of the surface.

The surface sealing can consist of or comprise various materials which on one hand are soluble in the given time and circumstances and on the other hand are biocompatible and tight. Thereby, the term “tight” can particularly relate to gas-tight or, more specifically, tight for contaminations such as deposits of organic (e.g., natural hydrocarbon molecules present in the atmosphere of cleanroom production facilities, as well as on work gloves in cleanrooms, and/or on production equipment in cleanrooms) or non-organic (e.g., residuals deriving from manufacturing processes such as electro-polishing) matter, dust, fibers, chemical impurities or particles in general.

Preferably, the surface sealing comprises a soluble carbohydrate, a soluble polymer, a soluble ionic compound or a combination thereof that does not directly interact with the surface other than sealing it. In experiments, sugars have shown a surprisingly advantageous capability to stably preserve target characteristics provided to surfaces of vascular devices. Accordingly, it has been discovered that a sugar film provided to cover the at least a portion of the surface together with the ions forms a sealing which shields from an interaction with contaminating agents. Sugars are also highly soluble and have exhibited an unexpected aptitude to adhere and remain attached to plain surfaces of vascular devices without peeling off, even under some applied mechanical stress and/or after a prolonged time (i.e., aging). Moreover, sugars have shown a pronounced elasticity and compliance to deformations, which is an advantageous attribute as it aptly supports flexibility and deformability of vascular devices during, e.g., mounting onto a delivery device, storage and/or vibrations during transportation/shipment, as is the preferred case for the present invention.

Thereby, the soluble carbohydrate can be a monosaccharide such as Glucose, Galactose, Fructose, Mannose or similar; a sugar alcohol such as Threitol, Erythritol, Sorbitol, Galactitol, Mannitol, Xylitol, Myo-inositol or similar; an organic acid such as citric acid, vitamin C or similar.

Or, the soluble carbohydrate can be a di- or a trisaccharide, such as Trehalose, Maltotriose, Lactose, Lactulose, Palatinose, Sucrose or similar.

For selecting an appropriate or optimal material, substance or sealing agent for the surface sealing plural factors of the given situation or application are to be taken into account. Considering what is required for sealing a surface of a vascular device, such as a stent or the like, regarding surface characteristics, sealing flexibility, clinical handling, quality assurance, manufacturing, sterilization, transportation, shelf life, etc., a homogenous, glass-like transparent surface sealing which is stable and fast soluble at the same time is typically desired or beneficial. For fast solubility and including also the above requirements, mono- and disaccharides and sugar-alcohols as mentioned above are well suitable. Also, a mix of such mono- and disaccharides with a salt or sugar-alcohol with a salt can be beneficial. For example, Trehalose in combination with a salt or ions has shown beneficial sealing and other properties. Larger molecules like tri-saccharides, such as e.g., Erlose, are typically not as beneficial due to their lower solubility which might make it difficult to be dissolved in the final application. However, in some applications, also such slower soluble tri-saccharide surface sealings might be beneficial.

In summary, the surface sealing may form a homogenous, glass-like transparent, gas-tight covering that is conformal to the vascular device contour and, for flexible implant structures, flexible. It also has good adhesion to the surface and/or to the delivery device (onto which the implant is mounted), has a good solubility (not too fast, not too slow such as, e.g., in the time range mentioned above), has a good drying behaviour (after drying not too brittle), is stable during sterilization such as by radiation or high temperature and/or high humidity ethylene oxide, is bio-compatible, has an easy regulatory pathway, and does not affect the functionalization of the surface by provision of the double or more charged ions.

Preferably, the surface sealing is seamlessly covering the functionalized surface. Like this, the surface can be uniformly covered and contamination can continuously be prevented. Further, the surface sealing preferably is gas-tight. Such surface sealing allows for efficiently preventing contamination of the surface through the atmosphere. Furthermore, as mentioned above, the at least a portion of the surface preferably is a plain surface which may lack a substantial roughness, waviness of its topology, any substantial texture, a coating, or a combination thereof. Still further, the at least a portion of the surface preferably has predefined target characteristics, wherein the predefined target characteristics preferably comprise hydrophilicity.

In another aspect, the invention is a method of manufacturing a vascular device for insertion in a body lumen. The method comprises the steps of (i) obtaining a vascular device with a surface; (ii) preparing at least a portion of the surface of the vascular device, and (iii) providing double or more charged ions to the at least a portion of the surface such that the at least a portion of the surface is functionalized.

With such a method, a vascular device as described above can be efficiently manufactured. Thereby, the effects and benefits described above in connection with the vascular device and its preferred embodiments can efficiently be achieved. In addition to that, the method according to the invention allows for providing the vascular device with preferred surface properties and functionalizing the surface by the provision of the at least double charged ions.

Thereby, step (iii) preferably comprises the sub-steps of (iii-1) obtaining an ion solution of the double or more charged ions, and (iii-2a) immersing the at least a portion of the surface in the ion solution, (iii-2b) spraying the ion solution on the at least a portion of the surface, or (iii-2c) rinsing the at least a portion of the surface with the ion solution. Also, combinations of these sub-steps are possible. Such provision of the ions to the surface via an ion solution allows for an efficient implementation. Thus, the vascular device functionalized with the ions at its at least a portion of the surface can efficiently be generated.

Step (iii) can further comprise a sub-step (iii-3) of drying the at least a portion of the surface. Thereby, the ions may stay on the surface whereas the solvent is removed, e.g., by elevating the temperature and removing the solvent. Particularly, the ions may be bound to the surface such that removal when drying the surface can be prevented.

Step (ii) of the method preferably comprises removing contaminants. It can also be a process in which contaminants are removed as a side effect, such as e.g., some kind of etching. Like this, a pure or plain surface can be generated which can be desired in many applications of vascular devices, such as when being implemented as stent or similar device. Thereby, removing contaminants preferably comprises plasma treatment, sterilization, etching, electro-polishing, or a combination thereof. Such procedures allow for an efficient and gentle removal of the contaminants.

Furthermore, step (ii) preferably comprises generating hydrophilicity. Such hydrophilicity may help to improve the functionalizing of the surface, e.g., binding the ions to the surface as described in (iii) and therefore to decrease the tendency to generate thrombi or the like.

In a preferred embodiment, the method comprises a step of (iv) covering the entire or at least a portion of the functionalized surface with a surface sealing which is soluble when inserting the vascular device in the body lumen. Thereby, step (iv) preferably comprises the sub-steps of (iv-1) obtaining a sealing solution of a sealing agent, (iv-2) immersing the entire or at least a portion of the functionalized surface in the solution, spraying the sealing solution on the entire or at least a portion of the functionalized surface, or rinsing the entire or at least a portion of the functionalized surface with the sealing solution, and (iv-3) drying the sealing agent on the functionalized surface. Such a method allows for efficiently providing a vascular device with preferred surface properties, a functionalized surface and a protection of the prepared and functionalized surface. Thereby, sub-step (iii-2) and sub-step (iv-2) can be combined in one single process step which allows for increasing the efficiency compared to a double drying procedure. The same applies for sub-step (iii-3) and sub-step (iv-3).

Preferably, the method comprises a step of (v) packaging and sterilising the vascular device by applying a radiation or a gas subsequently to covering the functionalized surface and the surface sealing.

The surface sealing can be embodied as explained above in connection with the vascular device according to the invention. Particularly, it can comprise a soluble or particularly water-soluble carbohydrate. Thereby, the soluble carbohydrate can be a monosaccharide such as Glucose, Galactose, Fructose, Mannose or similar; a sugar alcohol such as Threitol, Erythritol, Sorbitol, Galactitol, Mannitol, Xylitol, Myo-inositol or similar; an organic acid such as citric acid, vitamin C or similar. Or, the soluble carbohydrate can be a di- or a trisaccharide, such as Trehalose, Maltotriose, Lactose, Lactulose, Palatinose, Sucrose or similar.

Preferably, step (iv) comprises configuring the surface sealing to be dissolved when the functionalized surface is inserted to a target location in the body lumen.

Moreover, analogously to the vascular device according to the invention, within the method according to the invention the sealing can comprise ions preferably double or more charged such as, e.g., Phosphate ions. The at least a portion of the surface preferably is made of Nitinol, Stainless Steel, a Chromium alloy such as Cobalt-Chromium or Platinum-Chromium, Platinum-Chromium or a polymeric plastic; in a preferred embodiment, the surface is Nitinol functionalized with Phosphate ions. The vascular device preferably is a vascular stent, a flow diverter, an ocular stent, a coil or web-like structure for the treatment of vascular aneurysm, a heart valve, a cage of a heart valve, a part of a cardiac pacemaker such as an electrode, a flow disruptor, a web or web-like coil, a neck bridging device, an intra-aneurysmal stent, an occluder, an adjustable remodelling mesh, an aneurism clip, a vena cava filter, or a shunt; and/or the at least a portion of the surface of the vascular device preferably is a contact surface configured to contact a bodily fluid when the vascular device is inserted in the body lumen.

In a further other aspect, the invention is a use of a vascular device according to the invention or any of its preferred embodiments described above, comprising a step of making the vascular device ready for implantation by a pre-implantation preparation, and a step of implanting the made ready vascular device. Thereby, the pre-implantation preparation comprises flushing the vascular device with a bodily fluid or with an artificial or simulated bodily fluid. In particular, such flushing of the vascular device can be performed shortly before implantation of the vascular device for reducing the risk of contamination of the surface of the vascular device after flushing. The bodily fluid can be a bodily fluid as it is present at the location where the vascular device is implanted. For example, in many applications the bodily fluid is blood. Similarly, the artificial simulated bodily fluid can be a fluid to mimic the bodily fluid present at the location where the vascular device is implanted. In particular, the artificial simulated bodily fluid can be designed with specific properties and components as the bodily fluid present at the location where the vascular device is implanted. For example, the artificial simulated bodily fluid can be a balanced salt solution such as a Hanks' balanced salt solution or a serum such as blood serum.

By the specific flushing of the vascular device with the bodily fluid or the simulated bodily fluid shortly prior implantation, it can be achieved that the double or more charged ions of the functionalized surface of the vascular device recruit polyvalent ions from the bodily fluid or the artificial simulated bodily fluid. Like this, the vascular device can be provided with a stable antithrombotic surface. For example, when the functionalized surface comprises Phosphate ions and the vascular device is flushed with blood or an appropriate solution, it can be achieved that the pre-treated surface of the vascular device is stabilized with Calcium which has proven to have beneficial antithrombotic properties.

In still a further aspect, the invention is a use of a vascular device according to the invention or any of its preferred embodiments described above, comprising a step of implanting the vascular device, wherein flushing the vascular device prior to implantation is excluded. By excluding any flushing, it can be achieved that the functionalized surface of the vascular device is only flushed with the bodily fluid after implantation. Thereby, the formation of the mentioned antithrombotic or otherwise beneficial surface can be promoted after implantation.

By excluding the flushing prior to implantation, a particularly efficient procedure or use can be implemented. Specifically, in situations where at the location of implantation or insertion there is a sufficient amount or flow of bodily fluid accessible, such as implanting into a blood vessel or the like, this use may be beneficial.

In still another further aspect, the invention is a use of a vascular device according to the invention or any of its preferred embodiments described above, comprising a step of making the vascular device ready for implantation by a pre-implantation preparation, and a step of implanting the made ready vascular device, wherein the pre-implantation preparation comprises flushing the vascular device with a fluid preserving surface properties of the vascular device. The fluid preserving surface properties can be a fluid allowing to maintain the double or more charged ions on the surface of the vascular device. More specifically, the fluid can be configured to keep the double or more charged ions available on the surface such that functionalization of the surface can be maintained. Like this, it can be achieved that the surface properties and, particularly, the double or more charged ions are accessible after setting the vascular device to a target location. For preserving the surface properties, the fluid can contain double or more charged ions as well and, particularly, the same double or more charged ions as provided on the surface of the vascular device. For example, in embodiments of the vascular device being provided with Phosphate ions as charged ions, the fluid preserving surface properties can comprise Phosphate ions as well, e.g., the fluid can be a Phosphate ion solution and, specifically, a solution containing phosphate ions as the only anions.

By the specific flushing of the vascular device shortly prior to implantation with the fluid preserving the surface properties according to this use, it can be achieved that the double or more charged ions of the functionalized surface of the vascular device are kept or maintained until the vascular device is set to its target location, i.e., in a body of a patient. There, the double or more charged ions may recruit polyvalent ions from a bodily fluid, e.g., blood. Like this, the vascular device can be provided with a stable antithrombotic surface. For example, when the functionalized surface comprises Phosphate ions and the vascular device is flushed with a Phosphate ion solution, it can be achieved that the surface of the vascular device stays functionalized essentially until positioned at its target location, e.g., in a blood vessel or the like. The functionalized surface can be stabilized with Calcium which has proven to have beneficial antithrombotic properties.

Furthermore, in embodiments of the vascular device having a surface sealing, flushing the vascular device, e.g., by the bodily fluid, the simulated bodily fluid or the fluid preserving the surface properties, may at least partially remove or dissolve the surface sealing such that the plain functionalized surface is exposed when implanting or inserting the vascular device. Compared to removing the surface sealing after implantation, this may allow for a quicker formation of the mentioned antithrombotic surface.

In one specific example, an embodiment of the vascular device according to the invention is manufactured in an embodiment of a method according to the invention. Thereby, Phosphate ions are provided to the surface of a vascular device such as a vascular stent made of Nitinol (NiTi) or Titanium. The stent is prepared, i.e., purified, e.g., by plasma treatment, immersed in a Phosphate-containing solution, dried and preferably sealed.

X-ray Photoelectron Spectroscopy (XPS) investigation showed that Phosphate ions are present on NiT surface even after thorough rinsing processes with saline solution and deionized water. The control sample (not functionalized and not sealed) showed a surface Phosphate concentration below detection limit.

TABLE 1
Analysis results
Results XPS Analysis A18_0806

| | P concentration/at % | |
|---|---|---|
| Sample | (in form of Phosphate) | Rinsing |
| BO-18096_A | 4.0 | 5 × saline solution |
| BO-18096_B | 2.9 | 5 × saline solution & |
| | | 5 × deionized water |
| BO-17055_control | 0.4 (below detection | Not rinsed |
| | limit of 1%) | |

In vitro human blood loop tests show significant reduction of thrombotic reaction on functionalized and sealed NiT stents. This is performed for both, regular and surface-treated stents, i.e., bearing Phosphate ions on the surface. The stents are exposed to human blood in a blood loop flow model also called Chandler Loop. The regular (untreated) stent forms a thrombus whereas the thrombus formation on the surface-treated stent is significantly reduced and in some cases it remains even completely free from any thrombus.

The NT- or Titanium stent of the specific example provided with the Phosphate ions on its surface allows for recruiting bivalent ions and particularly Calcium ions from blood or a blood simulating fluid while or after being implanted. Like this, it can be achieved that the Calcium stabilizes the surface when being implanted. Such a surface has advantageous antithrombotic properties beneficial for the implanted stent. More specifically, by the stent of the specific example, it is possible to generate a stent surface upon implantation which is very difficult to achieve earlier since insoluble precipitates might form.

### BRIEF DESCRIPTION OF THE DRAWINGS

The vascular device according to the invention and the method of manufacturing a vascular device according to the invention are described in more detail herein below by way of exemplary embodiments and with reference to the attached drawings, in which:

FIG. 1 shows a vascular stent as a vascular device according to the invention;

FIG. 2 shows a surface of the vascular stent of FIG. 1 after preparation while being manufactured in a method according to the invention;

FIG. 3 shows the surface of FIG. 2 exposed to an ion solution while being manufactured;

FIG. 4 shows the surface of the vascular stent of FIG. 1 provided with ions while being manufactured;

FIG. 5 shows the surface of FIG. 4 exposed to a sealing solution while being manufactured;

FIG. 6 shows the surface of the vascular stent of FIG. 1 having a surface sealing after being manufactured;

FIG. 7 shows the surface of FIG. 6 contaminated with various substances;

FIG. 8 shows the surface of the vascular sent of FIG. 1 after the surface sealing is dissolved; and

FIG. 9 shows the surface of the vascular stent being implanted.

### DETAILED DESCRIPTION

In the following description of embodiments of the invention, to avoid repetition in the figures and the descriptions of the various aspects and illustrative embodiments, it should be understood that many features are common to many aspects and embodiments. Omission of an aspect from a description or figure does not imply that the aspect is missing from embodiments that incorporate that aspect. Instead, the aspect may have been omitted for clarity and to avoid prolix description. In this context, the following applies to the rest of this description: If, in order to clarify the drawings, a figure contains reference signs which are not explained in the directly associated part of the description, then it is referred to previous or following description sections. Further, for reason of lucidity, if in a drawing not all features of a part are provided with reference signs it is referred to other drawings showing the same part. Like numbers in two or more figures represent the same or similar elements.

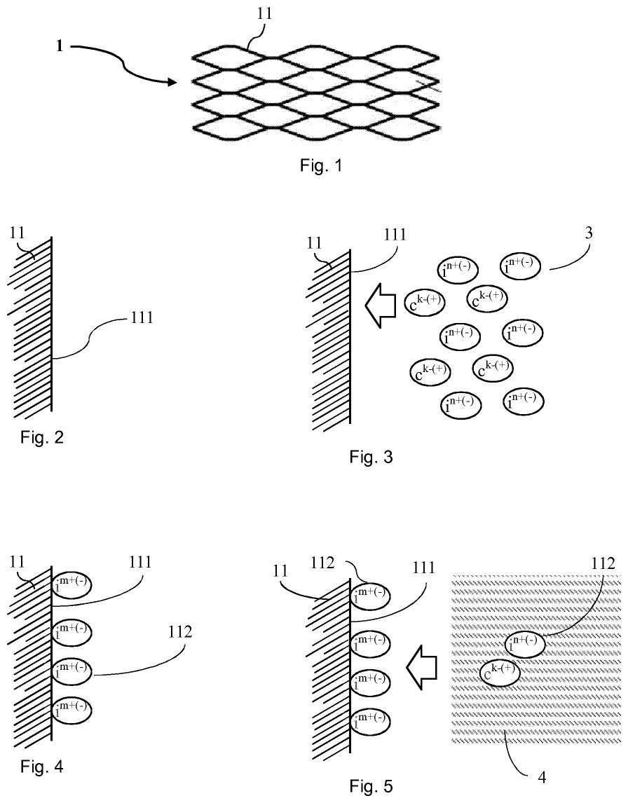

FIG. 1 shows a vascular stent 1 as an embodiment of a vascular device according to the invention. The stent 1 is manufactured in an embodiment of a method according to the invention as described in more detail below. In particular, the stent 1 is a self-expanding stent having a pattern of webs 11 forming dosed cells. More specifically, the multiplicity of webs 11 is made of Nitinol (NiTi) and the webs 11 together establish plural dosed cells, which form a tubular shape. The stent length and, as a passage, the stent lumen with a compressible diameter extend between a proximal and a distal end. The stent 1 has an expanded diameter in the dilated or released state, which is dimensioned for supporting a blood vessel as a body lumen into which the stent 1 is intended to be inserted or implanted.

As can be seen in FIG. 2, the webs 11 of the stent 1 establish a contact surface 111 of the stent 1 which is a portion of the complete surface of the stent 1 designed to contact blood as a bodily fluid when the stent 1 is inserted in a blood vessel. For example, the contact surface 111 is prepared by applying a plasma treatment in order to remove contaminants and generate a plain surface. Furthermore, within preparing the stent 1, hydrophilicity is generated on the contact surface 111.

In a next step shown in FIG. 3, the stent 1 is immersed in an ion (iⁿ⁺⁽⁻⁾) solution 3 including an inactive counter ion cm). Thereby, a (i^{m+(-)}) species 112 is bound to the contact surface 111 as can be seen in FIG. 4. This results, the contact surface being established as functionalized surface. In a preferred embodiment, the ion solution is a NaH₂PO₄ or KH₂PO₄ aqueous solution at pH 4.5, wherein the (iⁿ⁺⁽⁻⁾) ion 3 is mainly H₂PO₄⁻ and the ion (i^{m+(-)}) 112 is a Phosphate species bound to the contact surface 111 of Nitinol. Thus, in the preferred embodiment, the Nitinol contact surface is functionalized with Phosphate ions.

As depicted in FIG. 5, the stent 1 is then immersed again in a sealing solution 4. Such a sealing solution can comprise a sugar (such as e.g. Glucose, Trehalose, etc.) as a sealing agent and iⁿ⁺⁽⁻⁾ ions. The iⁿ⁺⁽⁻⁾ ions 3 as well as the inactive counter ions c^{k+(-)} can be contained in the sealing solution 4 in order to potentially prevent that the i^{m+(-)} ions 112 on the contact surface 111 are transferred into the sealing solution 4. In the preferred embodiment, the sealing agent is Trehalose.

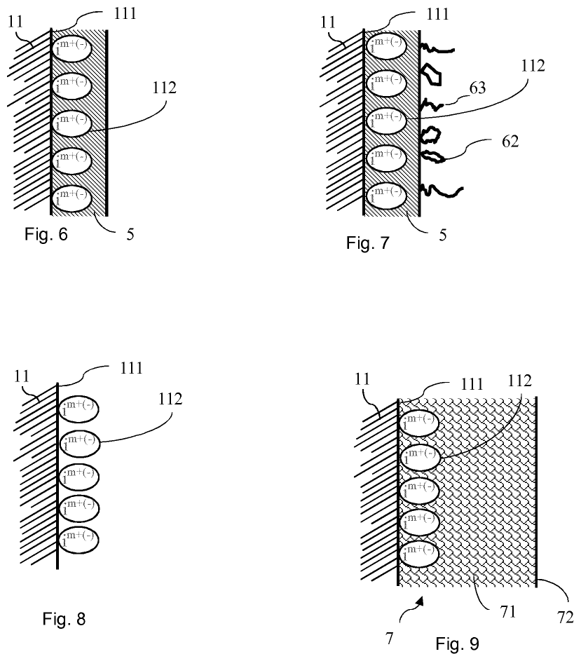

In a next step, the sealing solution is dried on the contact surface 111 such that a surface sealing 5 is generated covering the contact surface 111 with the i^{m+(-)} ion 112 as can be seen in FIG. 6. The pure hydrophilized contact surface 111 provided with the i^{m+(-)} ions 112, i.e., the functionalized surface, is thereby protected by the surface sealing 5.

FIG. 7 shows that in further processing, such as mounting onto the delivery device, packaging, sterilizing, storing and handling of the stent 1 before insertion into a body lumen, the surface sealing 5 is contaminated. In particular, hydrocarbon deposits or deposits of other undesired organic matter 62, machining impurities 63 such as fibers, dust, etc. can adhere to the sealing layer.

Before being inserted, the stent 1 and particularly its contact surface 111 is flushed by an appropriate liquid. Thereby, the sealing layer 5 is dissolved and removed together with the contaminants 62, 63 from the contact surface 111. As can be seen in FIG. 8, the highly purified plain contact surface 111 together with the i^{m+(-)} ions 112, i.e., the functionalized surface, is now in the same state as after the drying step of its manufacture (FIG. 4). The stent 1 is inserted into a blood vessel 7 and implanted at a target location thereof. FIG. 9 shows that the blood vessel 7 has a tubular shape with a wall 72. Inside the blood vessel 7 blood 71 flows. The contact surface 111 is directed towards the blood 71 such that the i^{m+(-)} ions 112 are exposed to the blood 71. Thereby, the i^{m+(-)} ions 112 reduce or prevent the adherence of thrombocytes onto the stent surface 111 inside the blood vessel 7.

This description and the accompanying drawings that illustrate aspects and embodiments of the present invention should not be taken as limiting-the claims defining the protected invention. In other words, while the invention has been illustrated and described in detail in the drawings and foregoing description, such illustration and description are to be considered illustrative or exemplary and not restrictive. Various mechanical, compositional, structural, electrical, ion charge, and operational changes may be made without departing from the spirit and scope of this description and the claims. In particular ions with their charges are purely illustrative and do not indicate actual charges. In some instances, well-known circuits, structures and techniques have not been shown in detail in order not to obscure the invention. Thus, it will be understood that changes and modifications may be made by those of ordinary skill within the scope and spirit of the following claims. In particular, the present invention covers further embodiments with any combination of features from different embodiments described above and below.

Even though the inventive concept is particularly useful or beneficial for vascular devices as described above, it can also be useful in other applications where the formation of thrombi or general foreign body reaction, such as e.g., inflammatory reaction, is to be prevented or reduced. In particular, surfaces of other implants or portions thereof can be functionalized by being provided with double or more charged ions. For example, in dentistry, often implants extend through the soft tissue or gingiva when being set into the bone of a jaw. Thereby, within the gingiva it can be desired to have as few thrombus formation, reduced inflammatory reactions or similar effects as possible. Thus, the sections of such implants which are designed to be located within the gingiva typically are prepared, such as polished or the like. Other sections of the surface of the implant are not polished or even roughened for allowing an efficient osseointegration. Applying the concept of the invention to such dental implants, the section to be located in the soft tissue can be functionalized by providing double of more charged ions. Also, other dental elements intended for being positioned in the gingiva such as abutments or healing caps can be surface treated accordingly. Additionally, other aspects described in connection with the vascular device according to the invention above can be applied to other implants when it is desired to reduce thrombus formation, inflammatory reactions or to achieve similar effects.

The disclosure also covers all further features shown in the Figs. individually although they may not have been described in the afore or following description. Also, single alternatives of the embodiments described in the figures and the description and single alternatives of features thereof can be disclaimed from the subject matter of the invention or from disclosed subject matter. The disclosure comprises subject matter consisting of the features defined in the claims or the exemplary embodiments as well as subject matter comprising said features.

Furthermore, in the claims the word “comprising” does not exclude other elements or steps, and the indefinite article “a” or “an” does not exclude a plurality. A single unit or step may fulfil the functions of several features recited in the claims. The mere fact that certain measures are recited in mutually different dependent claims does not indicate that a combination of these measures cannot be used to advantage. The terms “essentially”, “about”, “approximately” and the like in connection with an attribute or a value particularly also define exactly the attribute or exactly the value, respectively. The term “about” in the context of a given numerate value or range refers to a value or range that is, e.g., within 20%, within 10%, within 5%, or within 2% of the given value or range. Components described as coupled or connected may be electrically or mechanically directly coupled, or they may be indirectly coupled via one or more intermediate components. Any reference signs in the claims should not be construed as limiting the scope.

1. A vascular device for insertion in a body lumen, the device comprising:
a surface comprising at least a portion that is a functionalized surface provided with double or more charged ions such that the ions are exposed to a bodily fluid when the vascular device is inserted in the body lumen.

2. The vascular device of claim 1, wherein the ions are anions comprising phosphate, sulfate, borate or carbonate groups or organic acids, a combination thereof, or molecules with more than one charged group.

3. The vascular device of claim 1, wherein the ions are Phosphate ions, and/or the ions are bound to the at least a portion of the surface.

4. The vascular device of claim 1, wherein the at least a portion of the surface is made of Titanium, a Titanium alloy such as Nitinol, or a Chromium alloy such as Cobalt-Chromium or Platinum-Chromium, Tantalum, Platinum, or Zirconium oxide.

5. The vascular device of claim 1, wherein the device is a vascular stent, a flow diverter, an ocular stent, a coil or web-like structure for treatment of vascular aneurysm, a heart valve, a cage of a heart valve, a part of a cardiac pacemaker, a flow disruptor, a web or web-like coil, a neck bridging device, an intra-aneurysmal stent, an occluder, an adjustable remodelling mesh, an aneurism clip, a vena cava filter, or a shunt.

6. (canceled)

7. The vascular device of claim 1, wherein at least a portion of the functionalized surface is covered with a surface sealing which is soluble when inserting the vascular device in the body lumen, wherein the surface sealing is preferably configured to dissolve within 30 seconds.

8. (canceled)

9. The vascular device of claim 7, wherein the surface sealing is provided with double or more charged ions, wherein the ions of the surface sealing preferably are of the same type as the ions of the at least a portion of the surface.

10. (canceled)

11. The vascular device of claim 7, wherein:
the surface sealing comprises a soluble carbohydrate, a soluble polymer, a soluble ionic compound, or a combination thereof;
the soluble carbohydrate preferably is a monosaccharide such as Glucose, Galactose, Fructose, Mannose or similar; a sugar alcohol such as Threitol, Erythritol, Sorbitol, Galactitol, Mannitol, Xylitcol, Myo-inositol or similar; an organic acid such as citric acid, vitamin C or similar, or;
the soluble carbohydrate preferably is a di- or a trisaccharide, such as Trehalose, Maltotriose, Lactose, Lactulose, Palatinose, Sucrose or similar.

12. (canceled)

13. (canceled)

14. The vascular device of claim 7, wherein the surface sealing seamlessly covers the at least a portion of the surface, and/or the surface sealing is gas-tight.

15. (canceled)

16. The vascular device of claim 1, wherein the at least a portion of the surface is a plain surface which preferably lacks a substantial roughness, waviness of its topology, any substantial texture, a coating, or a combination thereof.

17. The vascular device of claim 1, wherein the at least a portion of the surface:
has predefined target characteristics, wherein the predefined target characteristics preferably comprise hydrophilicity; and/or
is a contact surface configured to contact the bodily fluid when the vascular device is inserted in the body lumen.

18. (canceled)

19. (canceled)

20. A method of manufacturing a vascular device for insertion in a body lumen, the method comprising
obtaining a vascular device with a surface;
preparing at least a portion of the surface of the vascular device; and
providing double or more charged ions to the at least a portion of the surface such that the at least a portion of the surface is functionalized.

21. The method of claim 20, wherein providing the double or more charged ions to the at least a portion of the surface comprises:
obtaining an ion solution of the double or more charged ions; and
immersing the at least a portion of the surface in the ion solution, spraying the ion solution on the at least a portion of the surface, or rinsing the at least a portion of the surface with the ion solution.

22. The method of claim 20, wherein preparing the at least a portion of the surface of the vascular device comprises:
removing contaminants, wherein removing contaminants preferably comprises plasma treatment, sterilization, etching, electro-polishing, or a combination thereof; and/or
generating hydrophilicity.

23. (canceled)

24. (canceled)

25. The method of claim 20, comprising covering at least a portion of the functionalized surface with a surface sealing which is soluble when inserting the vascular device in the body lumen, wherein covering the at least a portion of the functionalized surface with a surface sealing preferably comprises:
obtaining a sealing solution of a sealing agent;
immersing the at least a portion of the functionalized surface in the solution, spraying the sealing solution on the at least a portion of the functionalized surface, or rinsing the at least a portion of the functionalized surface with the sealing solution; and
drying the sealing agent on the functionalized surface.

26. (canceled)

27. The method of claim 25, comprising sterilising the vascular device by applying a radiation or a gas subsequently to covering the at least a portion of the surface together with the ions.

28. The method of claim 25, wherein the surface sealing comprises a soluble or particularly water-soluble carbohydrate, wherein the soluble carbohydrate preferably is:
a monosaccharide such as Glucose, Galactose, Fructose, Mannose or similar; a sugar alcohol such as Threitol, Erythritol, Sorbitol, Galactitol, Mannitol, Xylitol, Myo-inositol or similar; an organic acid such as citric acid, vitamin C or similar; or
a di- or a trisaccharide, such as Trehalose, Maltotriose, Lactose, Lactulose, Palatinose, Sucrose or similar.

29. (canceled)

30. (canceled)

31. The method of claim 25, wherein covering the functionalized surface with the surface sealing comprises configuring the surface sealing to be dissolved when the at least a portion of the surface is inserted to a target location in the body lumen.

32. The method of claim 20, wherein the ions are;
anions comprising phosphate, sulfate, borate or carbonate groups or organic acids, any combination thereof, or molecules with more than one charged group; and/or
Phosphate ions.

33. (canceled)

34. The method of claim 20, wherein the at least a portion of the surface is:
made of Titanium, a Titanium alloy such as Nitinol, a Chromium alloy such as Cobalt-Chromium or Platinum-Chromium, Tantalum, Platinum, or Zirconium oxide; and/or
a contact surface configured to contact a bodily fluid when the vascular device is inserted in the body lumen.

35. The method of claim 20, wherein the vascular device is a vascular stent, a flow diverter, an ocular stent, a coil or web-like structure for the treatment of vascular aneurysm, a heart valve, a cage of a heart valve, a part of a cardiac pacemaker such as an electrode, a flow disruptor, a web or web-like coil, a neck bridging device, an intra-aneurysmal stent, an occluder, an adjustable remodelling mesh, an aneurism clip, a vena cava filter, or a shunt.

36. (canceled)

37. A method of using a vascular device, wherein the device comprises a surface comprising at least a portion that is a functionalized surface provided with double or more charged ions such that the ions are exposed to a bodily fluid when the vascular device is inserted in the body lumen, the method comprising:
making the vascular device ready for implantation by a pre-implantation preparation, and implanting the made ready vascular device, wherein the pre-implantation preparation comprises flushing the vascular device with a bodily fluid or with a simulated bodily fluid; or
making the vascular device ready for implantation by a pre-implantation preparation, and implanting the made ready vascular device, wherein the pre-implantation preparation comprises flushing the vascular device with a fluid preserving the surface properties of the vascular device; or
implanting the vascular device, wherein flushing, the vascular device prior to implantation is excluded.

38. (canceled)

39. (canceled)
